(19) United States
(12) Patent Application Publication
(10) Pub. No.: US 2015/0039094 A1
(43) Pub. Date: Feb. 5, 2015

(54) CEMENT-FREE SURFACE REPLACEMENT PROSTHESIS FOR THE NATURAL FEMUR HEAD

(71) Applicant: CeramTec GmbH, Plochingen (DE)

(72) Inventor: Michael Kuntz, Homburg (DE)

(21) Appl. No.: 14/380,565

(22) PCT Filed: Feb. 12, 2013
(86) PCT No.: PCT/EP2013/052753

§ 371 Date: Aug. 22, 2014

(30) Foreign Application Priority Data
Feb. 24, 2012 (DE) 10 2012 202 833.1

(51) Int. Cl. A61F 2/36; A61F 2/30

(52) U.S. Cl. CPC A61F 2/3603; A61F 2/30771; A61F 2002/3085; A61F 2002/3093
USPC 623/23.14

(57) ABSTRACT

The invention relates to a ceramic surface replacement prosthesis (I) for the natural femur head (3), comprising an outer spherical surface (5) and an inner receiving space (7) with an inner lateral surface (4). The aim of the invention is to prevent necrosis of the bone tissue as a result of the implantation process, thus also preventing a loss of inner bone tissue. According to the invention, this is achieved in that the inner lateral surface (4) is provided with an osseointegrative coating (6) or is designed in an osseointegrative manner at least in some areas, and/or the lateral surface (4) is designed as a self forming thread (8).

The invention relates to a cement-free ceramic surface replacement prosthesis for the natural femoral head comprising an outer spherical surface and an inner receiving space having an inner lateral surface.

Surface replacement prostheses are also named resurfacing head, hip cup or cap prosthesis.

In cases of deterioration of the natural femur head or hip joint head, it is known in the state of the art to place a surface replacement prosthesis made from metal or ceramics on the femur head and anchor it down with bone cement. The surface replacement prosthesis replaces only the articular cartilage of the femoral head and is used when the femur bone under the articular cartilage is not substantially damaged in form or structure.

The outer surface of the surface replacement prosthesis has a spherical shape and is inserted either directly into the natural pelvis or into a synthetic socket, which in turn is anchored in the natural pelvis.

Up to now ceramic surface replacement prostheses have been based mainly on known metallic designs and are generally cemented in. The inner lateral surface of the internal receiving space is provided with cement pockets and a centering pin.

In view of the exothermic reaction during the hardening of the applied cement, there is danger of thermal damage and necrosis of the osseous tissue.

A cement-free version of a surface replacement prosthesis would be very advantageous for reason of the resulting heat generation of the cement.

Another advantage would be an implementation with no centering pin, as the loss of osseous tissue due to the consequent pin boring is diminished.

The object of the invention is to improve a ceramic surface replacement prosthesis according to the preamble of claim 1 in such a way that no necrosis of the osseous tissue is caused during implantation and at the same time no loss of internal osseous tissue occurs. Internal osseous tissue is that tissue which is not visible from the outside in contrast to outer osseous tissue, which is visible exteriorly and is treated before placement of the surface replacement prosthesis.

The object is achieved according to the invention, by providing the inner lateral surface at least in certain areas with an osseointegrative coating or by osseointegrative shaping and/or by forming the inner lateral surface as a self-forming thread.

An osseointegrative surface coating is described in B. Theelke, Mi. Kuntz, M. Zipperle, S. Eichhorm, T. Boxleiter, T. Pandorf, R. Burgkart, “Development of osseointegrative ceramic coatings based on ZPTA—mechanical characterization and influence on the substrate,” Bioceramics 1, 2010, pages 1-4.

By use of an osseointegrative surface coating or configuration of the inner lateral surface, a strong and long-lasting anchoring of the surface replacement prosthesis on the femoral head is achieved. An osseointegrative configuration of the inner lateral surface is realized, for example, by forming the near-the-surface area of the lateral surface in the incomplete state of the ceramic surface replacement prosthesis with the goal of obtaining a suitable open-pore structure.

Also a coating of the inner surface in the sintered state of the ceramic surface replacement prosthesis, for example, with structured coatings made from titanium alloys, is suitable.

An extremely important aspect of the invention is that the surface replacement prosthesis is fastened onto the femoral head without cement. This applies for all inventive embodiments. Also in all embodiments, no axial centering pin is disposed in the receiving space.

Alternatively or in combination with osseointegrative coating or osseointegrative shaping, the lateral surface is created as self-forming thread. In this way, the anchorage is substantially improved, so that cementation or the centering pin can be dispensed with.

The thread is formed with a conical or cylindrical shape. If the thread is conical, the femoral head as a rule must at least be worked on.

Surprisingly, it has been shown that the pitch of the thread exercises a considerable influence on the anchoring of the surface replacement prosthesis onto the head of the femur. The pitch of the thread is necessary to ensure that no loosening of the surface replacement prosthesis on the head of the femur occurs. The steeper the pitch is, the more forces are required to rotate the surface replacement prosthesis onto the head of the femur. Put in other words: for fixed surface replacement prostheses, if the torque acting via frictional forces on the surface replacement prosthesis is below the torque with which the surface replacement prosthesis is rotatable with its thread onto the femoral head, then the surface replacement prosthesis remains firmly anchored on the femoral head. The pitch of the thread is therefore an important feature of the invention.

It has been shown that the thread pitch is of essential importance for the primary fixation of the surface replacement prosthesis immediately after the operation, particularly to guaranty against loosening of the fixation due to an unwanted counter-clockwise rotation of the prosthesis: After the insertion of the head of the femur with surface replacement prosthesis into the acetabulum at the end of the operation, as a result of the natural ligament tension and muscle tone, the interface of the prosthesis with the head of the femur is axially pretensioned. The greater the steepness of the thread, the more torque is necessary for unwanted loosening of the junction; such a loosening torque can occur around the symmetry axis of the surface replacement prosthesis due to rotary movements of the leg during motion exercises. With increasing ingrowth of osseous tissue into the osseointegratively formed boundary layer between femoral head and surface replacement prosthesis, the positive connection between elements of osseous tissue (“bone trabeculae”) and the osseointegrative structure increases the stability of the interface.

Therefore the thread should be designed with a pitch greater than 2 mm/revolution. Even better is a pitch greater than 4 mm/revolution. The best results were obtained with a pitch greater than 6 mm/revolution. The thread can also be multi-start.

The number of thread turns, the tooth shape and the pitch can be optimized with respect to primary torsional reliability and axial stability within wide limits and are to be fabricated during the green machining of the ceramic surface replacement prosthesis on commercial CNC machines. Also very steep angles up to 90° are feasible, and these can be installed with an impact instrument.

The invention is further explained hereafter by means of several figures. Cement-free surface replacement prostheses are depicted in all figures.

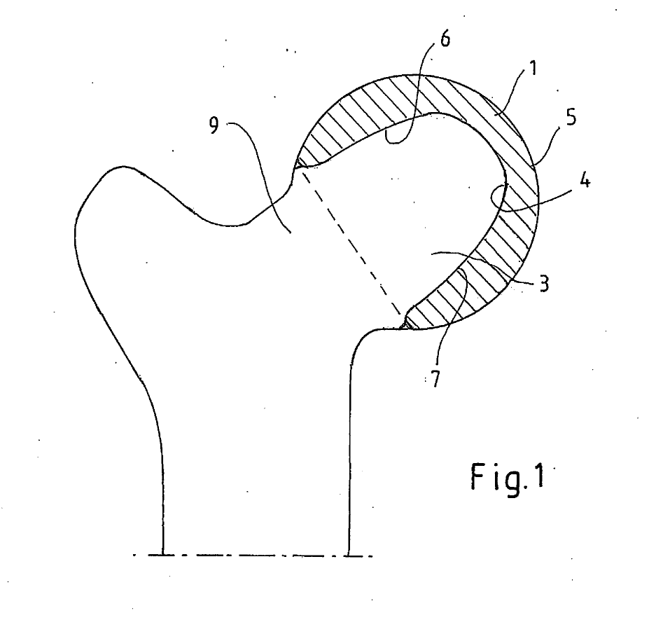

FIG. 1 schematically depicts a section through a natural femoral neck 9 and femoral head 3 with mounted surface replacement prosthesis 1 according to the invention made from a ceramic.

The outer surface of the prosthesis 1 is spherically formed and is either directly inserted into the natural pelvis or into a synthetic socket, which in turn is anchored directly or indirectly in the natural pelvis.

The surface replacement prosthesis 1 comprises an inner receiving space having an inner lateral surface 4, with which the surface replacement prosthesis 1 is fitted on the femoral head 3. The inner lateral surface 4 of the receiving space is preferably conically shaped. But the receiving space can also be ball-shaped, as shown in FIG. 1.

According to the invention, the surface replacement prosthesis 1 is fastened without cement onto the femoral head 3. This applies for all embodiments of the invention. Also, it is arranged with no axial centering pin in the receiving space.

To anchor the surface replacement prosthesis 1, the inner lateral surface 4 is provided with an osseointegrative coating 6 or is formed osseointegratively.

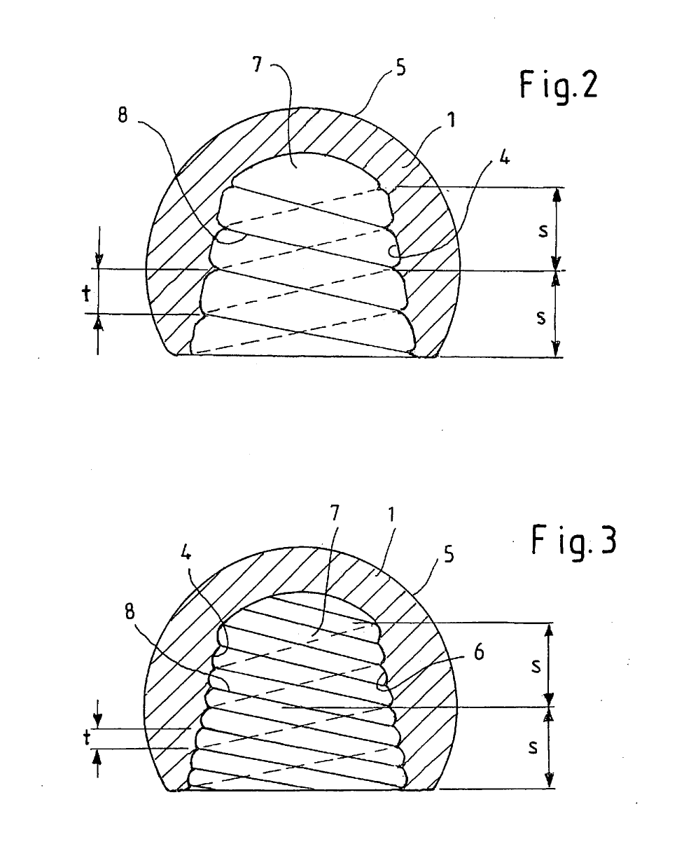

In another embodiment of the invention the inner lateral surface of the receiving space 4 is created as self-forming thread. These embodiments are explained in more detail by means of FIGS. 2-6. In addition, in these embodiments the inner lateral surface can also be provided with an osseointegrative coating or be created osseointegratively.

FIG. 2 illustrates a surface replacement prosthesis 1 according to the invention, in which the lateral surface 4 of the inner receiving space is formed in truncated cone shape and is provided with a multi-start high-pitch thread. A 2-start self-forming thread is depicted in FIG. 2 as an example. The inner lateral surface 4 is additionally provided with an osseointegrative coating 6 on the thread. An osseointegrative coating 6 is also designated as Direct-To-Bone-Coating. Preparation of the site during the operation takes place with an appropriately designed surgical milling instrument. After placement of the surface replacement prosthesis 1, this is fixed firmly by a right turn. To correct the height, the surface replacement prosthesis 1 can be loosened again by left rotation and if necessary the site can be re-milled.

Due to the centering effect of the cone-shaped site, a centering pin can be dispensed with the advantage of tissue retention.

In FIG. 2 the pitch(es) of the conical 2-start thread is constant, e.g. the axial path per total revolution is constant. The separation (t), e.g. the distance here between two thread tips is s/2.

FIG. 3 depicts a surface replacement prosthesis 1 with a conical self-forming 4-start thread with a separation s/4.

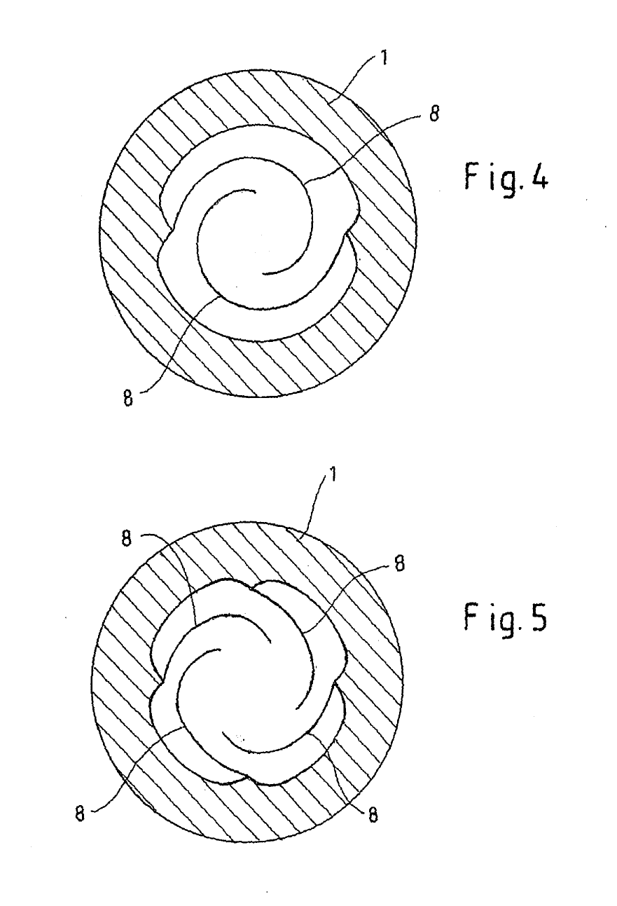

FIG. 4 depicts a section through a surface replacement prosthesis 1 with a 2-start thread 8 and FIG. 5 depicts a surface replacement prosthesis 1 with a 4-start thread.

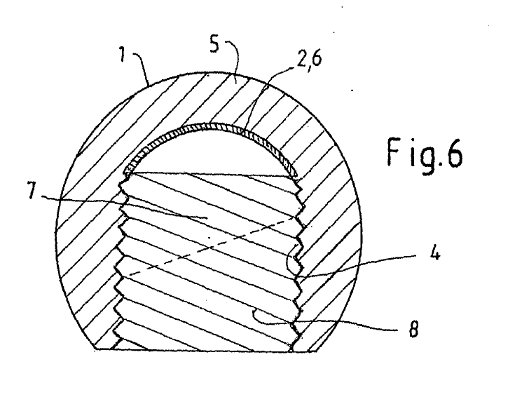

FIG. 6 depicts a surface replacement prosthesis 1 according to the invention with a cylindrical self-forming 6-start thread. Here only the spherical section 2 of the receiving space above the thread has an osseointegrative coating 6.

The pitch of all threads is 1 to 20 mm/revolution; preferred is 2 to 10 mm/revolution, and especially preferred is 3 to 6 mm/revolution. The choice of the start of the thread depends on the pitch and the thread form. Thus for a 1-start thread the pitch is preferably 1-2 mm/revolution, 2-4 mm/revolution for a 2-start thread and 3-6 mm/revolution for a 3-start thread.

### EXPLANATIONS FOR THE REFERENCE NUMERALS

1 surface replacement prosthesis
2 spherical section of the receiving space above the thread
3 femoral head
4 lateral surface
5 spherical surface
6 osseointegrative coating
7 receiving space
8 thread
9 femoral neck

1.-6. (canceled)

7. A ceramic surface replacement prosthesis for the natural femoral head comprising:
an outer spherical surface; and
an inner receiving space having an inner lateral surface,
wherein the inner lateral surface is provided at least in sectors with an osseointegrative coating or is osseointegratively formed.

8. A ceramic surface replacement prosthesis for the natural femoral head comprising:
an outer spherical surface; and
an inner receiving space having an inner lateral surface,
wherein the inner lateral surface is formed as self-forming thread.

9. A ceramic surface replacement prosthesis for the natural femoral head comprising:
an outer spherical surface; and
an inner receiving space having an inner lateral surface,
wherein the inner lateral surface is formed as self-forming thread; and
wherein the inner lateral surface is provided at least in sectors with an osseointegrative coating or is osseointegratively formed.

10. A surface replacement prosthesis according to claim 8, wherein the thread is conical or cylindrical in shape.

11. A surface replacement prosthesis according to claim 9, wherein the thread is conical or cylindrical in shape.

12. A surface replacement prosthesis according to claim 8, wherein the thread is formed with a pitch greater than 2 mm/revolution.

13. A surface replacement prosthesis according to claim 9, wherein the thread is formed with a pitch greater than 2 mm/revolution.

14. A surface replacement prosthesis according to claim 10, wherein the thread is formed with a pitch greater than 2 mm/revolution.

15. A surface replacement prosthesis according to claim 11, wherein the thread is formed with a pitch greater than 2 mm/revolution.

16. A surface replacement prosthesis according to claim 3, wherein the thread is formed with a pitch greater than 4 mm/revolution.

17. A surface replacement prosthesis according to claim 13, wherein the thread is formed with a pitch greater than 6 mm/revolution.

18. A surface replacement prosthesis according to claim 14, wherein the thread is formed with a pitch greater than 6 mm/revolution.

19. A surface replacement prosthesis according to claim 15, wherein the thread is formed with a pitch greater than 6 mm/revolution.

20. A surface replacement prosthesis according to claim 16, wherein the thread is formed with a pitch greater than 6 mm/revolution.

21. A surface replacement prosthesis according to claim 8, wherein the thread is formed multi-start.

22. A surface replacement prosthesis according to claim 9, wherein the thread is formed multi-start.

23. A surface replacement prosthesis according to claim 10, wherein the thread is formed multi-start.

24. A surface replacement prosthesis according to claim 11, wherein the thread is formed multi-start.

25. A surface replacement prosthesis according to claim 12, wherein the thread is formed multi-start.

26. A surface replacement prosthesis according to claim 13, wherein the thread is formed multi-start.
